(19) United States
(12) Patent Application Publication
(10) Pub. No.: US 2025/0387018 A1
(43) Pub. Date: Dec. 25, 2025

(54) MEDICAL DEVICES AND SYSTEMS WITH ARTICULATING SHAFTS

(71) Applicant: Boston Scientific Scimed, Inc., Maple Grove, MN (US)

(72) Inventors: John SUBASIC, Sutton, MA (US); Niraj Prasad RAUNIYAR, Plymouth, MN (US)

(73) Assignee: Boston Scientific Scimed, Inc., Maple Grove, MN (US)

(21) Appl. No.: 19/178,486

(22) Filed: Apr. 14, 2025

Related U.S. Application Data
(60) Provisional application No. 63/634,026, filed on Apr. 15, 2024.

(51) Int. Cl. A61B 1/005; A61B 1/00; A61B 1/05; A61B 1/06; A61M 25/01

(52) U.S. Cl. CPC A61B 1/0057; A61B 1/00114; A61B 1/0052; A61B 1/05; A61B 1/0676; A61M 25/0147

(57) ABSTRACT

Medical devices and systems with articulating shafts are described. The medical device may include a handle including an articulation mechanism, e.g., with an actuator configured to receive user input, and a shaft extending distally from the handle. The shaft may define an accessory channel that houses a composite cable extending from a proximal end of the shaft to a distal portion of the shaft. The composite cable may include at least one electrical wire operable coupled to at least one electronic component at or proximate a distal end of the shaft. The articulation mechanism may be configured to articulate the distal portion of the shaft by controlling movement of the composite cable by the user input received by the actuator.

### CROSS-REFERENCE TO RELATED APPLICATIONS

This application claims the benefit of priority to U.S. Provisional Application No. 63/634,026, filed on Apr. 15, 2024, which is incorporated by reference herein in its entirety.

### TECHNICAL FIELD

Aspects of the present disclosure generally relate to medical devices, system, and procedures related thereto. In particular, some aspects relate to medical systems and devices thereof with features for articulation during a medical procedure

### BACKGROUND

Medical devices such as ureteroscopes and endoscopes are useful for conducting medical procedures in a patient. In order to traverse through a patient's anatomy, the devices often have a reduced profile which limits the ability to incorporate features into the device, as well as the number and types of instruments that may be passed through a working channel of the device. Space at a distal face of the scope to accommodate electronic components such as imaging and illumination features is limited. Further, space within a cross section of a shaft of a medical scope to accommodate working channels, fluids channels, suction channels, electrical wires, and pull wires is similarly limited.

### SUMMARY

Each of the aspects disclosed herein may include one or more of the features described in connection with any of the other disclosed aspects.

The present disclosure includes medical devices and systems with articulating shafts. For example, the present disclosure includes a medical device comprising a handle including an articulation mechanism and a shaft extending distally from the handle. The articulation mechanism may include an actuator configured to receive user input and the shaft may define an accessory channel that houses a composite cable. The composite cable may extend from a proximal end of the shaft to a distal portion of the shaft, e.g., the composite cable including at least one electrical wire. The electrical wire(s) may be operably coupled to at least one electronic component at or proximate a distal end of the shaft. The articulation mechanism may be configured to articulate the distal portion of the shaft by controlling movement of the composite cable by the user input received by the actuator.

The medical device may include one or more of the following features. The at least one electronic component may include at least one of an imaging device or a light source, e.g., an imaging device and/or a light source at or near the distal end of the shaft. The at least one electrical wire may include a plurality of electrical wires. One or more of the plurality of electrical wires may be coupled to an imaging device and/or the light source. The composite cable may be anchored within the handle and/or within the shaft. For example, the articulation mechanism may include a cam within the handle, wherein the composite cable is anchored to the cam. A portion of the composite cable proximal to a location within the handle at which the composite cable is anchored may have slack. Additionally or alternatively, the composite cable may be anchored within the shaft proximate the distal end of the shaft, e.g., a portion of the composite cable distal to a location at which the composite cable is anchored having slack. Further, for example, the shaft may define at least one working channel extending from the proximal end of the shaft to the distal end of the shaft. The at least one working channel may be adjacent to the accessory channel. In some examples, the distal portion of the shaft may have a preformed curvature in a first direction. For example, the composite cable may be configured to move the distal portion of the shaft along a plane in a second direction opposite the first direction. According to some aspects, a distal portion of the handle may be rotatable relative to a proximal portion of the handle. Further, for example, the shaft may be rotatable with the distal portion of the handle relative to the proximal portion of the handle. The composite cable may include a cladding layer and/or insulation. For example, the cladding layer may form an outermost layer of the composite cable and/or the insulation may be around, e.g., radially surround, the at least one electrical wire (or plurality of electrical wires).

The present disclosure also includes a medical device. comprising a handle including an articulation mechanism comprising an actuator configured to receive user input and a shaft extending distally from the handle, the shaft defining an accessory channel that houses a composite cable extending from the handle and through a proximal end of the shaft to a distal portion of the shaft, the composite cable including a plurality of electrical wires operably coupled to at least one of an imaging device or a light source at a distal end of the shaft. The articulation mechanism may be configured to articulate the distal portion of the shaft by controlling movement of the composite cable by the user input received by the actuator. The composite cable may include a cladding layer. In some aspects, a proximal portion of the composite cable may be anchored within the handle and a distal portion of the composite cable may be anchored within the shaft, e.g., proximate the distal end of the shaft. The shaft may define at least one working channel extending from the proximal end of the shaft to the distal end of the shaft, e.g., the accessory channel being radially outward of the at least one working channel.

The present disclosure also includes a medical device comprising a handle including an articulation mechanism comprising an actuator configured to receive user input and a shaft extending distally from the handle, the shaft defining an accessory channel that houses a composite cable extending from the handle and through a proximal end of the shaft to a distal portion of the shaft, the composite cable including at least one electrical wire operably coupled to at least one electronic component at or proximate a distal end of the shaft; and at least one working channel adjacent to the accessory channel; wherein the articulation mechanism is configured to articulate the distal portion of the shaft by controlling movement of the composite cable by the user input received by the actuator; and wherein the composite cable is anchored within the handle and anchored within the shaft. For example, the handle may include a cam and the composite cable may be anchored to the cam.

### BRIEF DESCRIPTION OF THE FIGURES

The accompanying drawings, which are incorporated in and constitute a part of this application, illustrate exemplary aspects that, together with the written descriptions, serve to explain the principles of this disclosure. Each figure depicts one or more exemplary aspects according to this disclosure, as follows:

FIG. 1 depicts an exemplary medical device according to some aspects of the present disclosure.

FIG. 2 depicts a cross-sectional view of the medical device of FIG. 1.

FIG. 3 depicts a cross-sectional view of the medical device of FIG. 1.

FIG. 4 depicts a cross-sectional view of another exemplary shaft of a medical device according to some aspects of the present disclosure.

FIG. 5 depicts a cross-sectional of yet another exemplary shaft of a medical device according to some aspects of the present disclosure.

FIGS. 6 and 7 depict additional exemplary medical devices according to some aspects of the present disclosure.

### DETAILED DESCRIPTION

Particular aspects of the present disclosure are described in greater detail below. The terms and definitions provided herein control, if in conflict with terms and/or definitions incorporated by reference. The terms “proximal” and “distal” are used herein to refer to the relative positions of the components of exemplary medical devices. As used herein, “proximal” refers to a position relatively closer to the exterior of the body or closer to an operator using the medical device (see proximal “P” and distal “D” directional arrows in the figures). In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although ureteroscopes are referenced herein for illustration purposes, it will be appreciated that the disclosure encompasses any suitable medical device configured to allow an operator to access and view internal body anatomy of a subject (e.g., patient) and/or to deliver medical instruments, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the features described herein.

Aspects of the present disclosure provide medical devices with a working channel extending along at least a portion of a shaft to an opening at the distal tip, e.g., to facilitate delivery of instruments for treatment of a subject. The medical devices of the present disclosure include a composite cable including at least one electrical wire. For example, the composite cable may extend along an accessory channel of the shaft. The accessory channel may be adjacent to and/or radially outward of the working channel. The composite cable may be operatively coupled to an articulation portion of a shaft and configured to control articulation of a distal portion of the shaft, such that moving the composite cable in a direction may deflect the articulation portion of the shaft.

The medical device may be inserted into a body lumen and/or organ of a subject (e.g., into a bladder or kidney) in order to perform a medical procedure (e.g., lithotripsy, a biopsy collection, etc.). The distal tip of the medical device may include one or more electronic components, such as an imaging device and/or light source. For example, the medical device may include one or more or light sources such as light emitting diodes (LEDs), optical fibers (e.g., plastic optical fibers (POFs), other light guides, or a combination thereof), imaging devices (e.g., cameras, other components having imagers, and/or other optical elements such as lenses), and/or other electronic components (e.g., capacitors, diodes, resistors, etc.). According to some aspects herein, a distalmost face of the distal tip of the shaft may include an imaging device and/or a light source proximate the opening of the working channel at the distal end of the shaft.

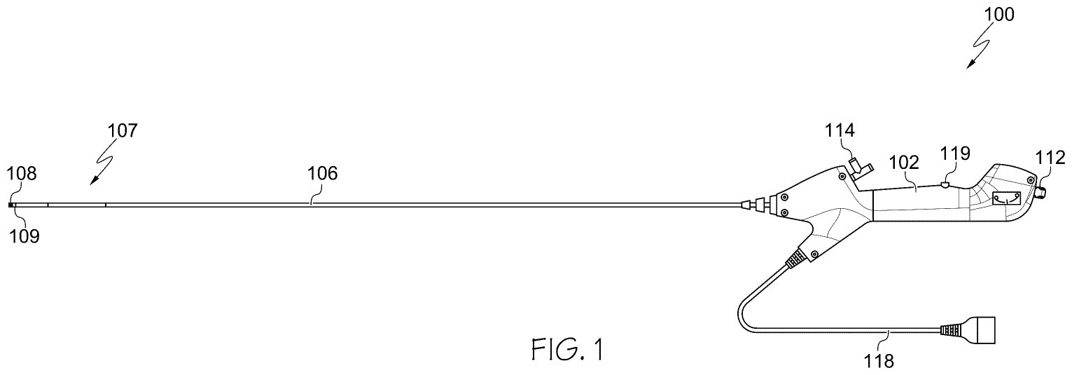

The following figures illustrate various features and examples of such medical devices. FIG. 1 depicts aspects of an exemplary medical device 100 that includes a handle 102 and a shaft 106. Handle 102 may be configured for gripping and use by an operator. Shaft 106 includes a distal portion 107, which terminates at a distal end 108. The distal end 108 may include an end cap 109.

At least a portion of shaft 106 may be flexible, for example, to facilitate navigation through a subject's anatomy. Shaft 106 may define one or more channels, the channel(s) may be configured to house or facilitate the passage of instruments and/or wires therethrough. The one or more channels may extend longitudinally through the shaft 106, e.g., from a proximal end of the shaft 106 to distal portion 107. In some examples, one or more of the channels terminates at the distal end 108.

The one or more channels of shaft 106 may include an accessory channel 110 configured to accommodate one or more wires or cables, such as a composite cable 140. Composite cable 140 may include at least one wire, e.g., at least one electrical wire operably coupled to at least one electronic component of medical device 100, and may be configured to articulate distal portion 107 of shaft 106 in one or more directions. The shaft 106 may also include a working channel 150 configured to receive an instrument extended therethrough. The instrument may include forceps, snares, clamps, scissors, electrocautery knifes, and other instruments useful for performing a medical procedure.

A port 114 of handle 102 may be in fluid communication with working channel 150. For example, an operator may insert one or more instruments into port 114 and extend the instrument(s) distally through working channel 150 to exit through a distal opening 150a.

Still referring to FIG. 1, an umbilicus 118 coupled to handle 102 may provide for electrical/electronic power to medical device 100. For example, umbilicus 118 may connect handle 102 to one or more user interfaces, monitors, controls units, displays, etc. for communication with one or more electronic components of medical device 100.

As mentioned above, composite cable 140 may include one or more wires, such as one or more electrical wires 142. The electrical wire(s) 142 may be electrically connected to a power source via umbilicus 118 and/or to a circuit board 144 within handle 102 or otherwise operably coupled to medical device 100 outside of handle 102. The electrical wire(s) 142 of composite cable 140 may be operably coupled to one or more electrical components 146 at or proximate the distal end 108 of shaft 106.

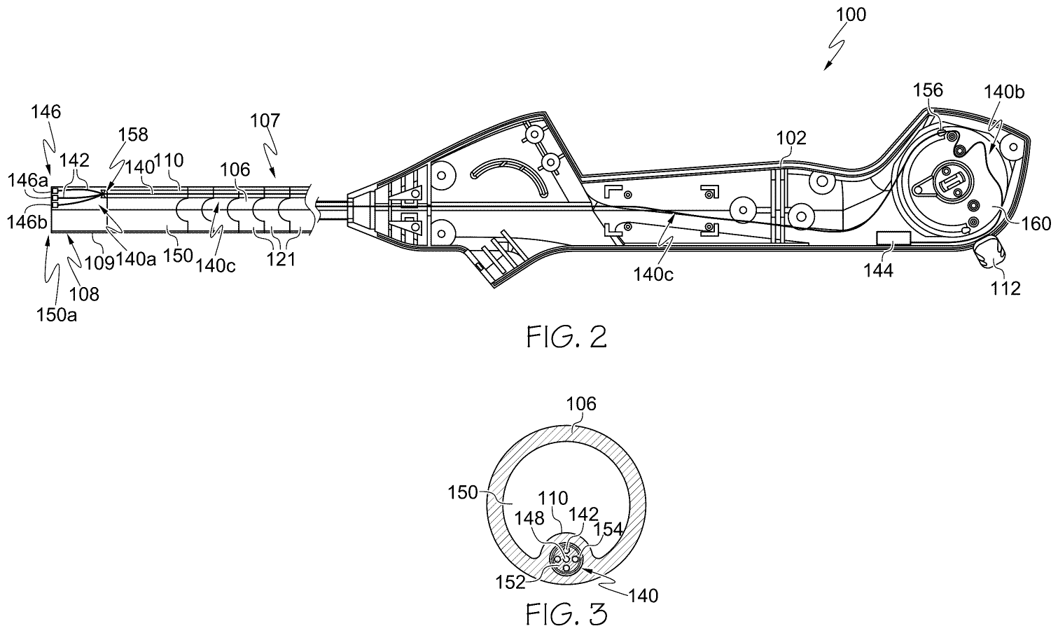

As mentioned above, shaft 106 may include one or more electronic components. For example, as illustrated in FIG. 2, distal end 108 of shaft 106 may include one or more light sources 146a (e.g., LED(s)) and one or more imaging devices 146b (e.g., camera(s)). Although two light sources 146a and one imaging device 146b are depicted in the figures, it will be appreciated that other combinations of light source(s) 146a and imaging device(s) 146b, as well as other types of electronic components, are encompassed by the present disclosure. Light sources 146a and imaging device 146b may be separate components or may be combined in single unit or device. Imaging device 146b may be configured to take video and/or still images. Imaging device 146b may provide a signal to a monitor or display (e.g., connected via umbilicus 118), so that an operator may view an image provided by imaging device 146b while navigating medical device 100 through a body lumen of a subject. Medical device 100 may be forward-facing. For example, features of distal end 108 (e.g., light source(s) 146a, and/or imaging device 146b) may face the same direction, such as, for example, a distal direction.

Handle 102 may include one or more actuators configured to control different features of medical device 100. The actuator(s) may be configured to receive and transmit user input. Exemplary actuators include, e.g., buttons, levers, switches, dials, etc. For example, handle 102 may include an actuator 119, illustrated in FIG. 1 as a button, operably coupled to imaging device 146b to capture images. While button 119 is shown on a proximal portion of handle 102, button 119 may be on another part of handle 102 accessible to the operator's hand during use. Handle 102 also may include an actuator 112, illustrated in FIG. 1 as a lever, operably coupled to an articulation mechanism for controlling movement of shaft 106.

Composite cable 140 may include a sufficient number of electrical wires 142 to supply power and/or signals to electronic components of medical device 100. For example, imaging device 146b may include multiple electrical connections to control various aspects of imaging device 146b, e.g., so that composite cable 140 includes at least two electrical wires 142 to be operably coupled to imaging device 146b. Composite cable 140 also may include one or more electrical wires 142 to supply power and/or signals to other electronic components, such as two additional electrical wires 142 each operably coupled to a corresponding light source 146a.

In addition to providing control over electronic components of medical device 100, composite cable 140 may be configured to control articulation of shaft 106. For example, composite cable 140 may be configured to function as a pull wire, such that moving composite cable 140 may correspond to movement of distal portion 107 of shaft 106. Optionally, composite cable 140 may include an articulation member such as a pull wire 148, e.g., adjacent to the one or more electrical wires 142 along the length of composite cable 140. For example, pull wire 148 may help to provide structural stability to composite cable 140. One or more electrical wires 142 may radially surround at least a portion of pull wire 148 (see, e.g., FIGS. 3-5).

As mentioned above, handle 102 may include an articulation mechanism to control articulation of medical device 100, e.g., the articulation mechanism including an actuator such as lever 112 operatively coupled to composite cable 140. For example, lever 112 may be coupled to composite cable 140 such that movement of lever 112 controls articulation of composite cable 140, which in turn may control articulation and steering of distal portion 107 of shaft 106. It will be appreciated that other suitable actuator(s) may be used in addition or in place of lever 112, such as one or more knobs, buttons, sliders, switches, or joysticks. Optionally, handle 102 may include one or more locking mechanisms, for example, to lock a position of lever 112 and, thus, lock an orientation of distal portion 107 and distal end 108 of shaft 106. As shown in FIG. 2, distal portion 107 may include one or more articulation segments 121 to facilitate deflection of distal portion 107.

According to some aspects of the present disclosure, composite cable 140 may include insulation and/or a cladding layer 152. For example, composite cable 140 may include a cladding layer 154, e.g., for structural integrity and/or to limit or prevent the transfer of electricity or signals outside composite cable 140. Cladding layer 154 may form an outermost layer of composite cable 140. Cladding layer 154 may comprise a polymer (e.g., rubber), textile, and/or metallic material. In at least one example, cladding layer 154 may comprise a braided sleeve of composite cable 140 (e.g., forming an outermost layer of composite cable 140). Additionally or alternatively, composite cable 14 may comprise insulation 152, e.g., between electrical wires 142 and/or pull wire(s) 148 within composite cable 140. Insulation 152 may comprise, for example, a polymer (e.g., rubber) or textile material. Optionally, insulation 152 may fill at least a portion or all space within composite cable between electrical wires 142 and/or pull wire(s) 148. Cladding layer 154 and/or insulation 152 may extend along only a portion or substantially an entire length of composite cable 140. A proximal portion and/or distal portion of composite cable 140 may be anchored within medical device 100. For example, anchoring composite cable 140 may permit slack to facilitate using the composite cable 140 to articulate the shaft 106 without disrupting electrical connections.

As illustrated in FIG. 2, in some examples, composite cable 140 may be anchored within shaft 106 proximal to distal end 108 and proximal to the one or more electronic components, e.g., light sources 146a and imaging device 146b. When shaft 106 includes an end cap 109, composite cable 140 may be anchored within end cap 109. Composite cable 140 may be anchored, for example, by a clamp, pinch, knot, or set screw, and/or may include an anchoring loop. For example, a portion of composite cable 140 may be anchored by forming a loop, one or more portions of the loop being anchored to an inner surface of shaft 106. Composite cable 140 may have slack distal to the distal anchoring location 158, e.g., wherein the one or more electrical wires 142 may connect to respective electronic components, e.g., light sources 146a and imaging device 146b. The distal anchoring location 158 may be a fixed location, for example, wherein composite cable 140 is fixed relative to shaft 106. A distal portion 140a of composite cable 140 (e.g., distal to the distal anchoring location 158) may remain in slack (e.g. a relaxed state) irrespective of articulation of shaft 106.

Further referring to FIG. 2, composite cable 140 may additionally or alternatively be anchored within handle 102, e.g., at a proximal anchoring location 156. Similar to distal anchoring location 158, composite cable 140 may be anchored within handle 102, for example, by a clamp, pinch, knot, or set screw, and/or an anchoring loop. For example, a portion of composite cable 140 may be anchored by forming a loop, one or more portions of the loop being anchored to an inner surface of handle 102. The proximal anchoring location 156 may be a fixed point, for example, wherein composite cable 140 is fixed relative to handle 102. In some examples, composite cable 140 may be anchored to a cam 160 within handle 102, e.g., cam 160 being rotatable to transfer user input received by lever 112 to movement of composite cable 140. In such cases, proximal anchoring location 156 may be on cam 160 and being movable within handle 102 together with cam 160. Cam 160 may be rotated clockwise or counterclockwise via actuation of lever 112. A proximal portion 140b of composite cable 140 may remain in slack irrespective of articulation of shaft 106, e.g., by lever 112. In some examples, proximal portion 140b may remain in slack irrespective of user input received by lever 112. The slack may prevent decoupling of electrical wire(s) 142 within handle 102 (e.g., from circuit board 144 within handle 102 and/or umbilicus 118). Proximal portion 140b may be of sufficient length so that proximal portion 140b is not in tension regardless of rotation of cam 160.

A middle portion 140c of composite cable 140 may maintain a constant length to permit articulation of shaft 106, e.g., via lever 112. Middle portion 140c may be held in tension between anchoring locations 156, 158 within handle 102 and shaft 106. For example, when lever 112 is actuated, middle portion 140c may be in tension.

FIGS. 2 and 3 depict additional aspects of medical devices according to the present disclosure. Any of the features described may be incorporated into one or more embodiments of medical device 100. Distal portion 107 of medical device 100 may include an actuated configuration and a deactuated configuration.

FIG. 3 depicts an exemplary cross-section of shaft 106, including accessory channel 110 and working channel 150. Accessory channel 110 may have dimensions suitable to accommodate composite cable 140. Working channel 150 may be configured to receive a medical instrument or may be used to pass fluid(s) therethrough and/or apply suction. Working channel 150 may be adjacent or otherwise proximate to accessory channel 110. Accessory channel 110 may be radially outward of working channel 150.

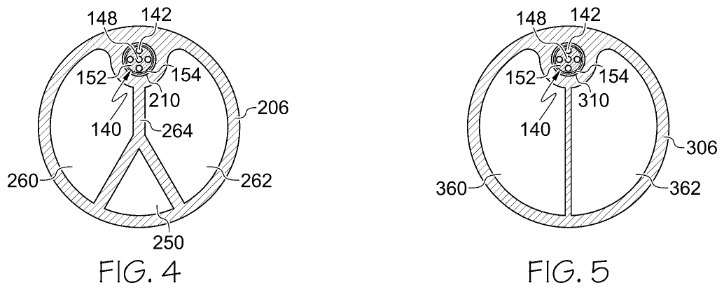

FIGS. 4 and 5 depicts additional exemplary cross section of shafts 206, 306 of medical devices according to the present disclosure. Shaft 206 and/or shaft 306 may be used in combination with any of the features of medical device 100 and/or shaft 106 of medical device 100.

As shown in FIG. 4, shaft 206 may include an accessory channel 210 and multiple working channels 250, 260, 262. Accessory channel 210 may include any of the features of accessory channel 110, e.g., suitable for accommodating composite cable 140. The working channels 250, 260, 262 may be used to introduce an instrument and/or fluid to a treatment site and/or to apply suction to a treatment site. For example, working channel 250 may be used for the passage of an instrument, working channel 260 may be configured to deliver fluid, and working channel 262 may be configured to provide negative pressure or suction. Shaft 206 may include a Y-shaped wall 264 to separate working channels 250, 260, 262 from one another along the length of shaft 206.

FIG. 5 depicts another exemplary shaft 306, including an accessory channel 310 and multiple working channels 360, 362. Accessory channel 310 may include any of the features of accessory channel 110 and/or 210, e.g., dimensions suitable for accommodating composite cable 140. The working channels 360, 362 may be used to introduce an instrument and/or fluid to a treatment site and/or to apply suction to a treatment site. For example, working channel 360 may be configured to deliver fluid and working channel 362 may be configured to apply suction.

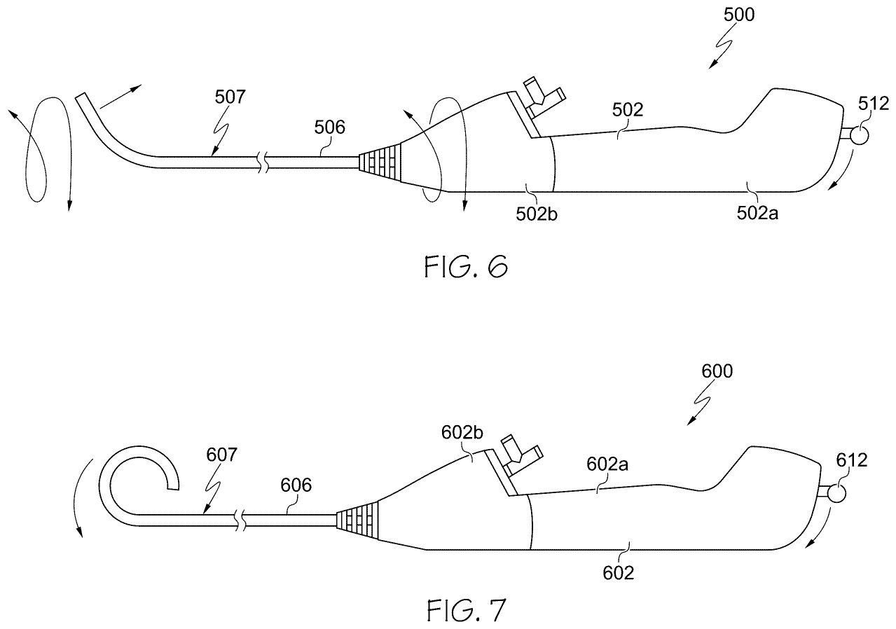

FIGS. 6 and 7 illustrate additional medical devices according to some aspects of the present disclosure with features for articulating the shafts of the respective medical devices.

For example, FIG. 6 shows medical device 500 that includes a handle 502 and a shaft 506. Handle 502 includes a proximal portion 502a and a distal portion 502b, proximal portion 502a including an actuator 512. As indicated by the directional arrows around distal portion 502b in FIG. 6, distal portion 502b may be rotatable relative to proximal portion 502a. Shaft 506 may be fixed to distal portion 502b such that rotating distal portion 502b relative to proximal portion 502a also rotates shaft 506. The ability to rotate shaft 506 and distal portion 502b of handle 502 relative to proximal portion 502a of handle may assist with navigating shaft 506 through a bodily lumen of a subject. In addition to rotation of shaft 506, actuator 512 may control deflection of a distal portion 507 of shaft 506 relative to a longitudinal axis of shaft 506 in a manner similar to actuator 112 of medical device 100. For example, medical device 500 may include any of the features of medical device 100, including, e.g., a composite cable similar to composite cable 140. Thus, for example, shaft 506 may include an accessory channel and one or more working channels as illustrated in FIG. 3, 4, or 5.

FIG. 7 shows medical device 600 that includes a handle 602 and a shaft 606. Handle 602 includes a proximal portion 602a and a distal portion 602b, proximal portion 602a including an actuator 612. In this example, a distal portion 607 of the shaft 606 has a preformed curvature in a first direction. The medical device 600 may include an articulation mechanism configured to articulate shaft 606, e.g., in a direction opposite the preformed curvature. For example, actuator 612 may control deflection of shaft 606 along a plane in a direction opposite the direction of the preformed curvature. Medical device 600 may include any of the features of medical device 100 and/or medical device 500. For example, shaft 606 may include a composite cable configured to move distal portion 607 in the direction opposite the preformed curvature. Shaft 606 may include an accessory channel and one or more working channels as illustrated in FIG. 3, 4, or 5.

The medical devices herein (e.g., medical devices 100, 500, 600) may be used during an exemplary medical procedure. For illustrative purposes, an exemplary procedure will be described using medical device 100. During the procedure, an operator may navigate shaft 106 through a bodily lumen so that distal end 108 is proximate a treatment site. The operator may deflect a distal portion of shaft 106 by actuating an actuator (e.g., lever 112) of medical device 100. User input received by the actuator may move composite cable 140 within accessory channel 110 (or accessory channel 210 or 310; see FIGS. 6 and 7) of the shaft 106 in a proximal direction and which may deflect the distal portion of the shaft 106 relative to the longitudinal axis of the shaft 106.

After reaching the treatment site, the operator may employ one or more electronic components at or proximate the distal end 108 of shaft 106 to visualize the treatment site. For example, the operator may engage an actuator such as button 119 to power and control light source(s) 146a and/or imaging device 146b. Additionally or alternatively, the operator may introduce a medical instrument or fluid through a working channel of shaft 106 and/or employ aspiration through a working channel of shaft 106 (see, e.g., working channels 150, 250, 260, 262, 360, and 362 in FIGS. 4, 6, and 7). After visualizing and/or delivering treatment to the treatment site, the operator may withdraw the shaft 106 from the subject.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Each of the aspects disclosed herein may include one or more of the features described in connection with any of the other disclosed aspects. Accordingly, the claimed features are not to be considered as limited by the foregoing description.

What is claimed is:

1. A medical device comprising:
a handle including an articulation mechanism comprising an actuator configured to receive user input; and
a shaft extending distally from the handle, the shaft defining an accessory channel that houses a composite cable extending from a proximal end of the shaft to a distal portion of the shaft, the composite cable including at least one electrical wire operably coupled to at least one electronic component at or proximate a distal end of the shaft;
wherein the articulation mechanism is configured to articulate the distal portion of the shaft by controlling movement of the composite cable by the user input received by the actuator.

2. The medical device of claim 1, wherein the at least one electronic component includes at least one of an imaging device or a light source.

3. The medical device of claim 1, wherein the at least one electrical wire includes a plurality of electrical wires.

4. The medical device of claim 3, wherein the medical device includes an imaging device at or near the distal end of the shaft, and wherein one or more of the plurality of electrical wires is coupled to the imaging device.

5. The medical device of claim 4, wherein the medical device includes a light source at or near the distal end of the shaft, and wherein one or more electrical wires of the plurality of electrical wires is coupled to the light source.

6. The medical device of claim 1, wherein the composite cable is anchored within the handle.

7. The medical device of claim 6, wherein the articulation mechanism includes a cam within the handle, and the composite cable is anchored to the cam.

8. The medical device of claim 6, wherein a portion of the composite cable proximal to a location within the handle at which the composite cable is anchored has slack.

9. The medical device of claim 1, wherein the composite cable is anchored within the shaft proximate the distal end of the shaft.

10. The medical device of claim 9, wherein a portion of the composite cable distal to a location at which the composite cable is anchored has slack.

11. The medical device of claim 1, wherein the shaft defines at least one working channel extending from the proximal end of the shaft to the distal end of the shaft, the at least one working channel being adjacent to the accessory channel.

12. The medical device of claim 1, wherein the distal portion of the shaft has a preformed curvature in a first direction, and the composite cable is configured to move the distal portion of the shaft along a plane in a second direction opposite the first direction.

13. The medical device of claim 1, wherein a distal portion of the handle is rotatable relative to a proximal portion of the handle, the shaft being rotatable with the distal portion of the handle relative to the proximal portion of the handle.

14. The medical device of claim 1, wherein the composite cable includes a cladding layer forming an outermost layer of the composite cable and insulation within the cladding layer, wherein the insulation surrounds the at least one electrical wire.

15. A medical device comprising:
a handle including an articulation mechanism comprising an actuator configured to receive user input; and
a shaft extending distally from the handle, the shaft defining an accessory channel that houses a composite cable extending from the handle and through a proximal end of the shaft to a distal portion of the shaft, the composite cable including a plurality of electrical wires operably coupled to at least one of an imaging device or a light source at a distal end of the shaft,
wherein the articulation mechanism is configured to articulate the distal portion of the shaft by controlling movement of the composite cable by the user input received by the actuator.

16. The medical device of claim 15, wherein the composite cable includes a cladding layer.

17. The medical device of claim 15, wherein a proximal portion of the composite cable is anchored within the handle and a distal portion of the composite cable is anchored within the shaft proximate the distal end of the shaft.

18. The medical device of claim 15, wherein the shaft defines at least one working channel extending from the proximal end of the shaft to the distal end of the shaft, the accessory channel being radially outward of the at least one working channel.

19. A medical device comprising:
a handle including an articulation mechanism comprising an actuator configured to receive user input; and
a shaft extending distally from the handle, the shaft defining:
an accessory channel that houses a composite cable extending from the handle and through a proximal end of the shaft to a distal portion of the shaft, the composite cable including at least one electrical wire operably coupled to at least one electronic component at or proximate a distal end of the shaft; and
at least one working channel adjacent to the accessory channel;
wherein the articulation mechanism is configured to articulate the distal portion of the shaft by controlling movement of the composite cable by the user input received by the actuator; and
wherein the composite cable is anchored within the handle and anchored within the shaft.

20. The medical device of claim 19, wherein the handle includes a cam, and the composite cable is anchored to the cam.
